(19) United States
(12) Patent Application Publication
(10) Pub. No.: US 2012/0041313 A1
(43) Pub. Date: Feb. 16, 2012

(54) ULTRASONIC IMAGING DEVICE

(75) Inventors: Tomohiko Tanaka, Hachioji (JP); Kunio Hashiba, Tokyo (JP); Mariko Yamamoto, Kokubunji (JP); Osamu Mori, Tokyo (JP)

(73) Assignee: HITACHI MEDICAL CORPORATION, Tokyo (JP)

(21) Appl. No.: 13/265,773

(22) PCT Filed: Apr. 23, 2010
(86) PCT No.: PCT/JP2010/057203

§ 371 Date: Oct. 21, 2011

(30) Foreign Application Priority Data
Apr. 24, 2009 (JP) 2009-106872

(51) Int. Cl. A61B 8/14

(52) U.S. Cl. 600/443

(57) ABSTRACT

The absolute pressure inside the heart with respect to heartbeat time phase is measured non-invasively or with minimal invasion. An ultrasonic diagnostic device comprising: a pressure sensor that detects artery pressure non-invasively; a reference pressure computation part that converts the artery pressure into absolute reference pressure with respect to a reference point; a spatial pressure difference calculation part that calculates a spatial pressure difference between the reference point and a location distinct from the reference point; and an absolute pressure computation part that calculates intracardiac absolute pressure using a shape image, the reference pressure, and the spatial pressure difference.

### TECHNICAL FIELD

The present invention relates to a medical ultrasonic imaging device, and, more particularly, to an ultrasonic imaging device that chronologically measures intracardiac absolute pressure as desired by the examiner.

### BACKGROUND ART

In many advanced countries, heart disease is one of the three leading causes of death. In making an early diagnosis of or monitoring heart disease, temporal pressure information with respect to the left atrium or the left ventricle is used as an index that is directly helpful for diagnostic purposes. The term pressure information as used above refers to differential pressure with respect to atmospheric pressure and will hereinafter be referred to as absolute pressure.

When measuring intracardiac absolute pressure, a method is employed whereby a cardiac catheter is inserted into the body. Information that may be obtained with a catheter is mainly absolute pressures with respect the aorta, the left ventricle and the left atrium, and fluctuations in absolute pressure caused by pulsation, that is, an absolute pressure waveform. This method is an invasive method where a cardiac catheter is inserted into the body and intracardiac pressure is measured directly.

In addition, as a non-invasive technique relating to measuring intracardiac pressure, there has been devised a method where blood velocity inside the heart is measured, and intracardiac pressure difference is calculated from the measured blood velocity using physical equations. The term pressure difference as used above refers to the pressure difference between two given points. More particularly, with respect to methods for calculating pressure difference from blood velocity, the following methods, which differ in how velocity is detected, have been reported. The method of Patent Document 1 measures a unidirectional component of a fluid having three-dimensional motion using the ultrasonic Doppler effect, and infers the three-dimensional behavior of the fluid using numerical calculations. In addition, the method of Non-Patent Document 1 measures a unidirectional component of a fluid having three-dimensional motion using the ultrasonic Doppler effect, and calculates a two-dimensional flow velocity vector by imposing an assumption of two-dimensional behavior. The methods of Patent Document 1 and Non-Patent Document 1 measure only a unidirectional velocity component of a fluid, and estimates other directional components. Thus, the pressure difference calculated based on the estimated flow velocity vector is effective for flow-fields with little three-dimensional influence. In addition, in Patent Document 2, high-precision two-dimensional blood velocity vectors are detected by tracking, over time, reflected signals from a contrast agent called Echo PIV.

As methods of measuring an absolute pressure waveform, there are methods that convert a radial main artery pressure waveform into a central aortic pressure waveform using a transfer function. In Non-Patent Document 2 and Non-Patent Document 3, a central aortic pressure waveform estimated from a radial main artery pressure waveform is compared with an actual central aortic pressure waveform, and favorable correspondence is demonstrated therebetween.

### PRIOR ART DOCUMENTS

### Patent Documents

Patent Document 1: JP 2004-121735 A
Patent Document 2: WO 2007/136554 A1

### Non-Patent Documents

Non-Patent Document 1: Tanaka, M. et al., Journal of Cardiology, 52, 86-101 (2008)
Non-Patent Document 2: Pauca, A. L., et al., Hypertension 38:932-937 (2006)
Non-Patent Document 3: Millasseau S. C., et al., Hypertension 41:1016-1020 (2003)

### SUMMARY OF THE INVENTION

### Problems to be Solved by the Invention

However, when a cardiac catheter is used, while it is possible to chronologically measure intracardiac absolute pressure, because it is an invasive measurement, the strain on the patient is considerable. In addition, with respect to methods that calculate intracardiac pressure difference from blood velocity using physical equations, since quantities that may be calculated through physical equations are relative pressure differences between two given points, absolute pressure cannot be measured. While pressure waveform measuring methods that use a transfer function are capable of chronologically measuring absolute pressure, they are restricted to aortic pressure. Application of transfer function methods to intracardiac pressure results in significant errors, and offers no precision with respect to diagnosability.

An object of the present invention is to measure non-invasively, or with minimal invasion, absolute pressure inside the heart at a desired location with respect to heartbeat time phase.

### Means for Solving the Problems

With the present invention, artery pressure is non-invasively and chronologically detected by means of a pressure sensor, and artery pressure is converted into absolute reference pressure inside the heart or at a reference point in proximity thereto at a given time phase by means of a transfer function. In addition, blood velocity is detected based on an ultrasonic imaging signal, and a spatial pressure difference between the reference point and a pressure calculation location, which is set inside the heart, is calculated based on blood velocity using the laws of physics. Further, using reference pressure and spatial pressure difference, intracardiac absolute pressure is calculated. In so doing, by changing the pressure difference calculation method depending on the heartbeat time phase, a continuous display of absolute pressure with respect to any given heartbeat time phase, that is, detection of an intracardiac absolute pressure waveform that is more precise than it has conventionally been, is made possible.

### Effects of the Invention

With the present invention, relative to the conventional examples where intracardiac pressure difference is measured based on fluid behavior, by calculating the absolute pressure of a reference part with good precision, it is possible to provide absolute pressure that is effective for diagnostic purposes. In addition, by virtue of the chronological measurements by the pressure sensor, it is possible to detect chronological pressure fluctuations of the heartbeat. Further, it is possible to provide an ultrasonic imaging device that chronologically measures intracardiac absolute pressure non-invasively or with minimal invasion.

### BRIEF DESCRIPTION OF THE DRAWINGS

FIG. 1A is a block diagram showing the device configuration of an ultrasonic imaging device of an embodiment of the present invention.

FIG. 1B is a block diagram showing the device configuration of an ultrasonic imaging device of an embodiment of the present invention.

FIG. 2 is a flowchart showing the operations of a signal processing part.

FIG. 3 is a flowchart showing the details of step S12.

FIG. 4 is a flowchart showing the details of step S13.

FIG. 5 shows charts illustrating heartbeat time phases with respect to intracardiac absolute pressure and aortic pressure.

FIG. 6 shows diagrams illustrating the opening/closing of heart valves with respect to heartbeat time phase.

FIG. 7(a) is a diagram illustrating Bernoulli's principle when the valve is closed, and (b) is a diagram illustrating Bernoulli's principle when the valve is open.

FIG. 8 is a diagram illustrating a state where a tracer has entered the heart.

FIG. 9(a) is an illustrative diagram where a tracer image is divided in a grid-like fashion, (b) is a diagram illustrating the tracking of changes in the tracer image over time, and (c) is a diagram illustrating velocity vectors as derived via the tracer.

FIG. 10 is a diagram illustrating the calculation of pressure difference as derived from velocity vectors.

FIG. 11 is a diagram illustrating the derivation of pressure difference from in-flow propagation velocity.

FIG. 12 is a diagram illustrating the changing of pressure difference calculation methods incorporating heartbeat time phases.

FIG. 13 is a diagram illustrating ROI settings for valve flow velocity detection.

FIG. 14(a) is a diagram showing a display screen for heartbeat time phase fluctuations in intracardiac absolute pressure and aortic pressure, (b) is a diagram showing a contour line display screen for intracardiac pressure and aortic pressure, and (c) is a diagram showing a display screen for a pressure-volume relationship diagram.

### MODES FOR CARRYING OUT THE INVENTION

Embodiments of the present invention are described below based on the drawings.

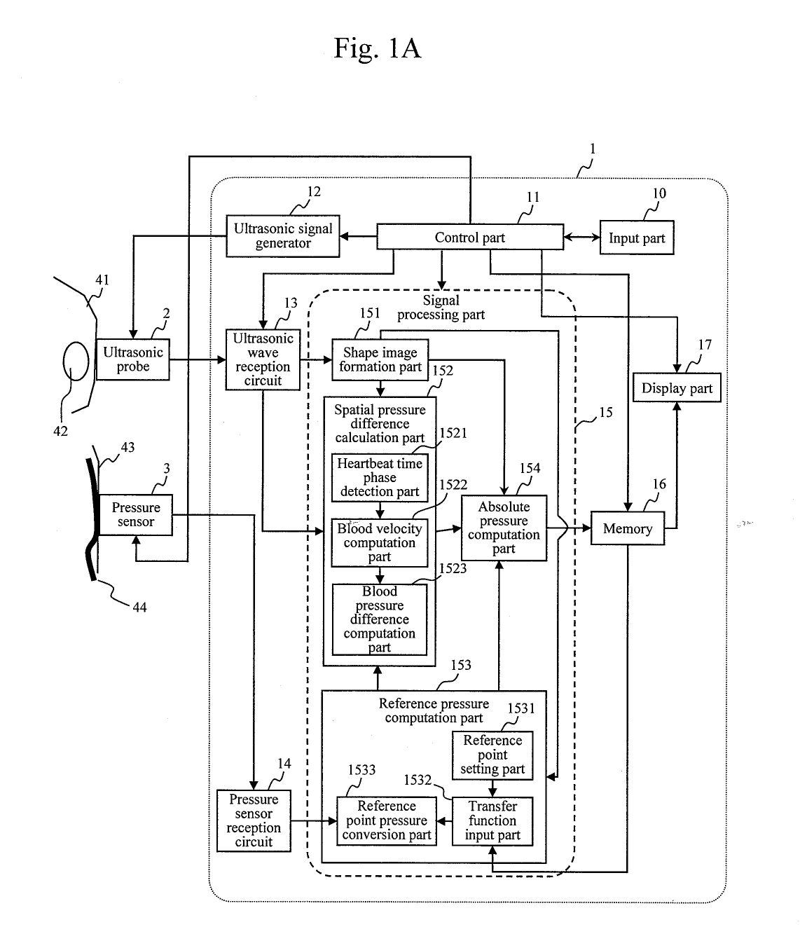

FIG. 1A is a block diagram showing a device configuration example of an ultrasonic imaging device according to the present invention. An ultrasonic imaging device of the present invention comprises a device main body 1, an ultrasonic probe 2, and a pressure sensor 3.

The device main body 1 controls the ultrasonic probe 2, while at the same using a blood pressure signal from the pressure sensor 3 to generate an ultrasound image. In accordance with a signal generated at an ultrasonic signal generator 12, the ultrasonic probe 2 comes into contact with a living organism (an examinee) 41, and irradiates an irradiation region 42 with an ultrasonic wave, while also receiving a reflected-wave echo signal of the irradiation region 42. The pressure sensor 3 measures the blood pressure of an artery 44 at a given site 43 of the living organism.

Next, detailed elements of the device main body 1 will be described. The device main body 1 comprises an input part 10, a control part 11, the ultrasonic signal generator 12, an ultrasonic wave reception circuit 13, a pressure sensor reception circuit 14, a signal processing part 15, memory 16, and a display part 17.

The input part 10 is a keyboard or a pointing device with which the examiner operating the ultrasonic imaging device sets operation conditions for the ultrasonic imaging device with respect to the control part 11, or an electrocardiography signal input part in cases where electrocardiography is employed. The control part 11 is a part that, based on the operation conditions for the ultrasonic imaging device set by the input part 10, controls the ultrasonic signal generator 12, the ultrasonic wave reception circuit 13, the pressure sensor reception circuit 14, the signal processing part 15, the memory 16, and the display part 17. By way of example, it is a CPU of a computer system. The ultrasonic wave reception circuit 13 performs signal processing, such as amplification, rectification, etc., on a reflected echo signal received by the ultrasonic probe 2. The pressure sensor reception circuit 14 converts a signal obtained from the pressure sensor 3 into pressure information and hands it over to the signal processing part 15. The signal processing part 15 has a function of generating an ultrasound image based on the reflected echo signal from the ultrasonic probe 2 and on the blood pressure signal from the pressure sensor 3. The memory 16 stores various information, namely the reflected echo signal, and the ultrasound image and blood pressure signal obtained at the signal processing part 15. The memory 16 also stores information that is held at an absolute pressure computation part 154 and at a blood velocity computation part 1522. The display part 17 outputs information that is stored on the memory 16.

Next, detailed elements of the signal processing part 15 will be described. The signal processing part 15 comprises a shape image formation part 151, a spatial pressure difference calculation part 152, a reference pressure computation part 153, and an absolute pressure computation part 154. Based on the reflected echo signal outputted from the ultrasonic wave reception circuit 13, the shape image formation part 151 forms, by way of example, a B-mode image, that is, an organ shape of the examinee.

The spatial pressure difference calculation part 152 comprises a heartbeat time phase detection part 1521, the blood velocity computation part 1522, and a blood pressure difference computation part 1523. The blood velocity computation part 1522 calculates blood velocity based on the reflected echo outputted from the ultrasonic wave reception circuit 13. With respect to a reference point obtained at a reference point setting part 1531 and to a given spatial point from the organ shape formed at the shape image formation part 151, the blood pressure difference computation part 1523 calculates the pressure difference relative to the reference point. Further, the heartbeat time phase detection part 1521 detects the heartbeat time phase based on the reflected echo outputted from the ultrasonic wave reception circuit 13. Heartbeat time phase detection may be carried out through, by way of example, recognition of the flow velocity direction passing through the valve by the blood velocity computation part 1522, or through recognition of the valve's opening/closing based on a flow velocity direction shape image, or through recognition of the heartbeat time phase based on an electrocardiography signal imported via the input part 10, and so forth.

The reference pressure computation part 153 comprises the reference point setting part 1531, a transfer function input part 1532, and a reference point pressure conversion part 1533. The reference point setting part 1531 sets a reference point based on the organ shape obtained at the shape image formation part 151. The transfer function input part 1532 reads out from the memory 16 a transfer function corresponding to the reference point that has been set at the reference point setting part 1531. The reference point pressure conversion part 1533 calculates the absolute pressure at the reference point based on the artery pressure information handed over from the pressure sensor reception circuit 14 and on the transfer function.

Based on the reference point absolute pressure obtained at the reference pressure computation part 153 and on the spatial pressure difference relative to the reference point at a given location as obtained at the spatial pressure difference calculation part 152, the absolute pressure computation part 154 calculates the absolute pressure of the given location.

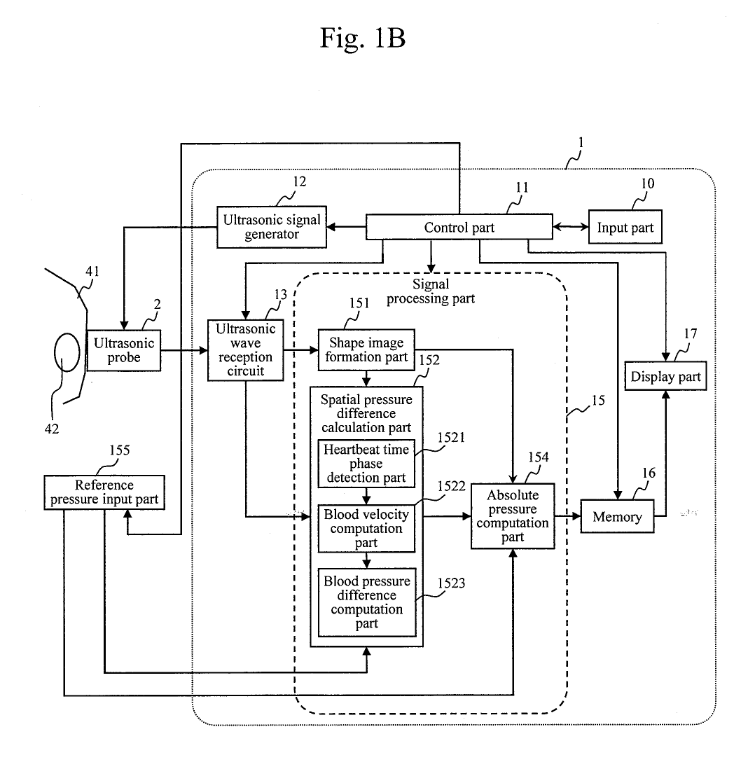

A process flow of the present embodiment is shown in FIG. 2. In FIG. 2, as a specific example, it is assumed that the irradiation region 42 in FIG. 1A is a site including the heart and the ascending aorta, that the given site 43 is the forearm, and that the artery 44 is the radial artery. First, the shape image formation part 151 converts an ultrasonic signal into, by way of example, a shape image for a living organism shape such as the heart and the aorta (S11), and sends the shape image to the reference pressure computation part 153 and the absolute pressure computation part 154. Next, the reference pressure computation part 153 converts the pressure obtained at the pressure sensor 3 to reference pressure P₀ of reference point X₀ (S12). Next, the spatial pressure difference calculation part 152 calculates the pressure difference between reference point X₀ and location X₁ (S13). Finally, the absolute pressure computation part 154 calculates intracardiac absolute pressure based on reference pressure P₀ and the spatial pressure difference (S14). Thus, through the processes at the reference pressure computation part 153, the spatial pressure difference calculation part 152 and the absolute pressure computation part 154, it becomes possible to obtain intracardiac absolute pressure based on radial artery pressure and an intracardiac blood velocity field. It is noted that step 12 and step 13 may be reversed in order or executed simultaneously.

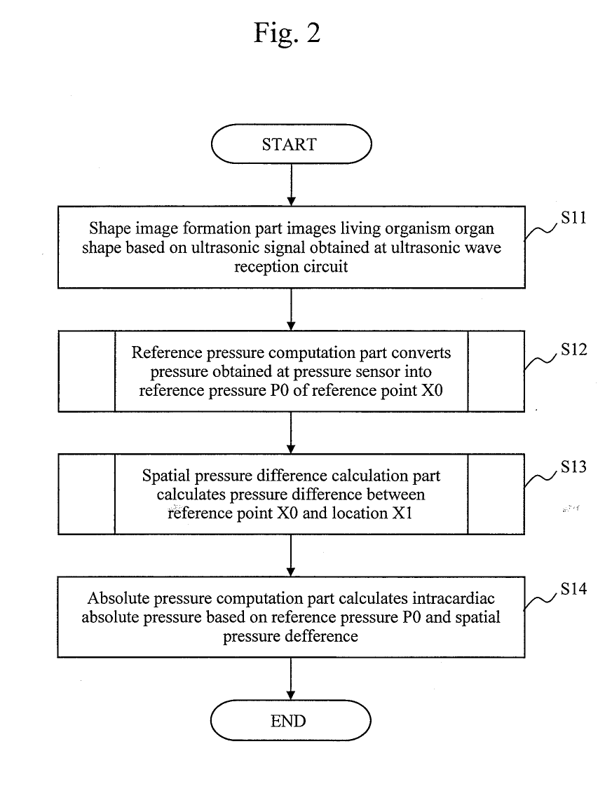

Next, a detailed process of the reference pressure computation part in step 12 will be described using FIG. 3. An image of the heart and the aorta is obtained from the shape image formation part 151 (S121). Next, at the reference point setting part 1531, based on the above-mentioned obtained image, the user sets reference point X₀ at, by way of example, a center part of the ascending aorta, which is representative of the ascending aorta. Although X₀ indicates the interior of the aorta in this case, it may also be a representative point within the left ventricle. Whether the reference point is to be set in the left ventricle or the aorta is decided by the user. It is noted that the setting of X₀ may also be performed by automatically detecting a reference organ shape calculated at the shape image formation part 151 (S122). The transfer function input part 1532 reads out from the memory 16 a transfer function corresponding to the reference point that has been set at the reference point setting part 1531. The reference point pressure conversion part 1533 calculates the absolute pressure with respect to the reference point based on the artery pressure information handed over from the pressure sensor reception circuit 14 and on the transfer function.

The transfer function input part 1532 reads out, from the memory 16 storing transfer functions, a transfer function corresponding to the reference point that has been set as mentioned above and to the site to be measured with the pressure sensor (S123). The transfer function is a function representing the relationship between phase and gain for a radial artery pressure waveform and an aorta pressure waveform with respect to a frequency space in which the radial artery pressure waveform and aorta pressure waveform, which are fluctuations in radial artery pressure and aorta pressure over time, are each Fourier transformed. The transfer function is phase and gain information per frequency, and phase and gain information is stored on memory. In addition, specific examples of transfer functions are also described in Non-Patent Document 3. Next, the pressure of the radial artery measured by the pressure sensor 3 is inputted (S124), and the reference point pressure conversion part 1533 converts the above-mentioned inputted pressure information to ascending aorta pressure P₀, which has been set as the reference point, based on the above-mentioned obtained transfer function (S125). Here, by having the pressure sensor employ tonometry, radial artery pressure with good precision is calculated. The transfer function is a function representing the relationship between phase and gain for the radial artery and aorta.

Alternatively, reference pressure P₀, e.g., ascending aorta pressure, etc., that has been set as the reference point may also be inputted via external input. A configuration diagram for one such case is shown in FIG. 1B. A reference pressure input part 155 inputs reference pressure P₀, e.g., ascending aorta pressure, etc., and communicates information on reference pressure P₀ to the spatial pressure difference calculation part 152 and the absolute pressure computation part 154.

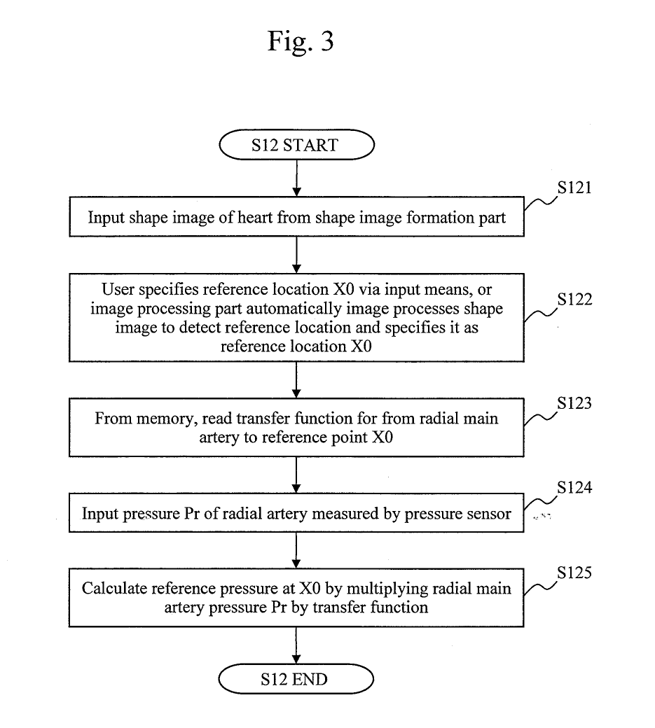

Next, a detailed process of the spatial pressure difference calculation part in step 13 will be described using FIG. 4. First, reference point X₀ that has been set as discussed above is inputted (S131). The image of the heart and aorta from the shape image formation part 151 is inputted (S132). Next, based on the obtained image discussed above, the user sets arbitrary location X₁ (S133). In the present case, X₁ is set as an arbitrary point inside the heart. It is noted that the setting of X₁ may be performed automatically through image processing by defining, for example, a center part inside the heart, etc., as being a representative site. In addition, X₁ may be a plurality of points, and the space may have two or more dimensions. Further, the heartbeat time phase detection part 1521 detects the heartbeat time phase based on an ultrasonic signal obtained from the ultrasonic wave reception circuit 13 (S134), and the pressure difference calculation method is determined (S135). The method for calculating the pressure difference inside the heart is determined in accordance with the state of valve opening or valve closure inside the heart. If the valve is closed, the regurgitant flow velocity at the location of the valve is detected, and Bernoulli's principle is selected as the pressure difference calculation method (S136). In addition, if the valve is open, the flow velocity at the location of the valve is detected, and the Navier-Stokes equation is selected (S137). In step 138, pressure difference ΔP between reference point X₀ and location X₁ that have been set in step 131 and S133 is calculated using the method selected in step 136 or step 137.

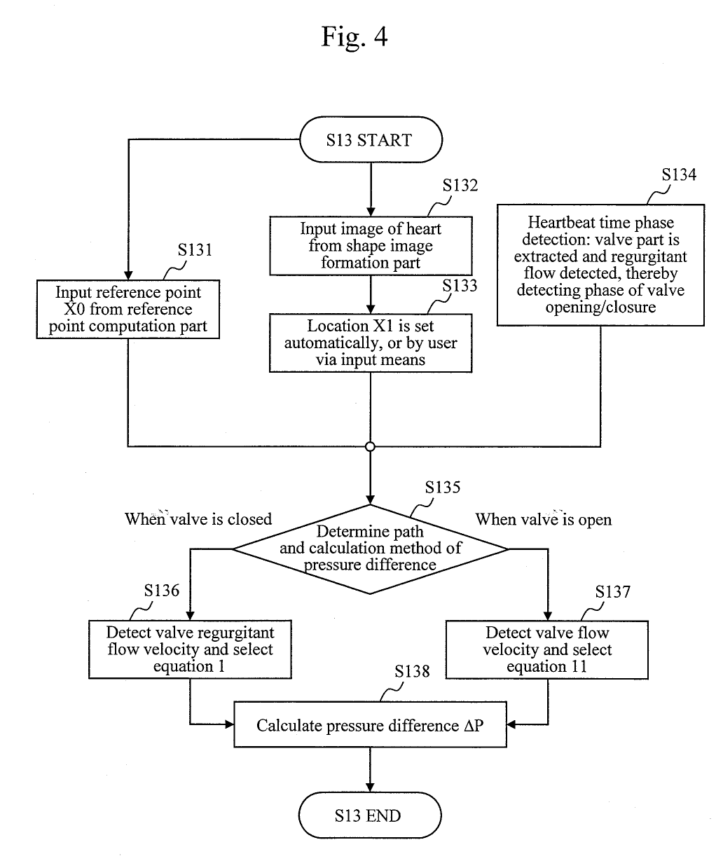

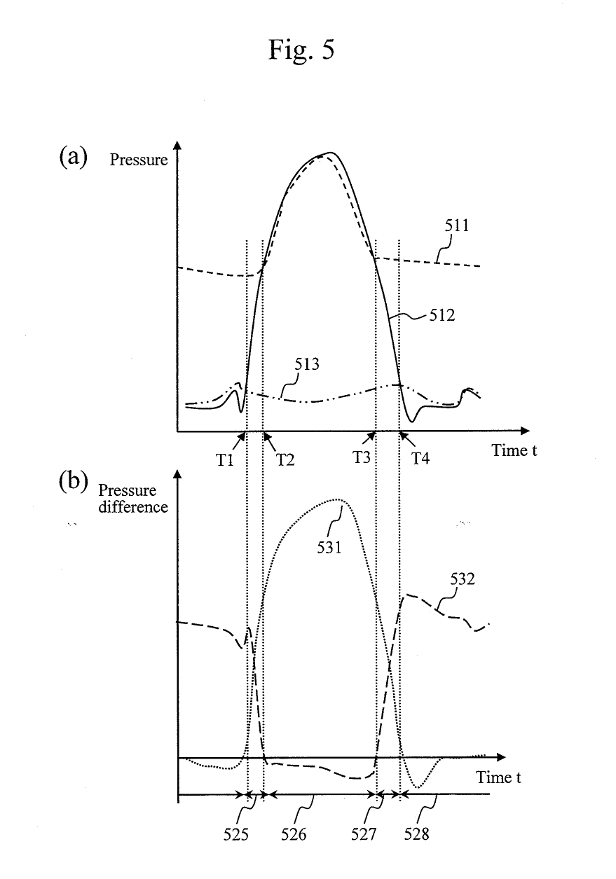

Details of the method of determining the pressure difference calculation method as carried out in step 135 will now be described using FIG. 5. The graph in FIG. 5(a) shows examples of pressure fluctuations over time with respect to one heartbeat. 511 represents pressure fluctuation in the aorta, 512 pressure fluctuation in the left ventricle, and 513 pressure fluctuation in the left atrium. In addition, schematics of changes that the heart undergoes over the course of one heartbeat are shown in FIG. 6. 61 represents the aorta, 62 the left atrium, 63 the left ventricle, 64 the aortic valve, and 65 the mitral valve.

The period from T1, which is the point at which the mitral valve closes, up to T2, which is the point at which the aortic valve opens, is called the isovolumetric contraction phase 525. The heart during this period of time is such that, as shown in FIG. 6(a), the aortic valve 64 and the mitral valve 65 are closed. At this point, at the aortic valve 64 and the mitral valve 65, an aortic valve regurgitant flow 641, which is leakage from a gap in the closed aortic valve, and a mitral valve regurgitant flow 651, which is leakage from a gap in the closed mitral valve, are taking place. The period from T2 up to T3, which is the point at which the aortic valve closes, is called the ejection phase 526. The heart during this period is such that, as shown in FIG. 6(b), the aortic valve 64 is open and the mitral valve 65 is closed. At this point, at the aortic valve 64 and mitral valve 65, an aortic valve forward flow 642 and the mitral valve regurgitant flow 651 are taking place. The period from T3 up to T4, which is the point at which the mitral valve opens, is called the isovolumetric relaxation phase 527, and as shown in FIG. 6(c), the aortic valve 64 and the mitral valve 65 are closed. At this point, at the aortic valve 64 and the mitral valve 65, the aortic valve regurgitant flow 641 and the mitral valve regurgitant flow 651 are taking place. Further, the period from T4 up to T1 of the subsequent heartbeat is called the filling phase 528, and as shown in FIG. 6(d), the aortic valve 64 is closed and the mitral valve 65 is open. At this point, at the aortic valve 64 and the mitral valve 65, the aortic valve regurgitant flow 641 and a mitral valve forward flow 652 are taking place.

For valve regurgitant flows, pressure difference may be calculated based on Bernoulli's principle. However, with respect to valve forward flows, Bernoulli's principle does not hold, and the pressure difference computation method needs to be changed. Although details will be discussed later, the computation method changing time is one or more of the times at which the state of the valve that lies in the path between reference point X₀ and location X₁ changes from closed to open or from open to close, namely, T1, T2, T3 and T4. The combination of reference point X₀ and location X₁, which serve as changing locations, is such that reference point X₀ is within the aorta 61 or within the left ventricle 63, and location X₁ within one of the left ventricle 63, the left atrium 62 and the aorta 61.

With regard to detecting a changing time, detection may be carried out as the time at which at least one of the following occurs: with respect to a B-mode image detected by the shape image formation part 151, the time at which the valve opens or closes, as well as the time at which the left ventricular volume or area becomes smallest or greatest, or the time at which a period during which the greatest or smallest state is sustained begins or ends, as well as, with respect to an M-mode image, the time at which the valve opens or closes, as well as the time at which a sign reversal occurs with respect to the blood velocity detected by the blood velocity computation part 1522. Here, the term B-mode image refers to an image representing an organ shape as imaged via ultrasound, and the term M-mode image refers to an image that temporally represents organ movement by tracking organ movement along a given ultrasound scanning line over time, and representing the position of the organ along the scanning line with the vertical axis and time with the horizontal axis.

Next, details of the pressure difference calculation methods will be discussed. First, the pressure difference calculation method for when a valve regurgitant flow is detected while a valve is closed will be discussed. When a valve regurgitant flow is detected, pressure difference may be calculated using Bernoulli's principle. For a valve regurgitant flow, it may be a detection method that utilizes the Doppler effect, or a method that tracks blood cells or a pre-administered tracer, e.g., contrast agent, etc., within the regurgitant blood through image recognition. As a simplified method of Bernoulli's principle that utilizes regurgitant velocity, there is the simplified Bernoulli equation. Assuming the regurgitant velocity is V, pressure difference ΔP in and out of the valve may be expressed through the equation below.

ΔP=A×V² (1)

where A is a constant equal to or greater than 3.5 but equal to or less than 4.5 and whose unit is [sec²·mmHg].

As this equation contains an assumption of a steady state, the unsteady Bernoulli equation indicated below, which takes unsteady influences into account, may be used as well. B is a term that unsteady influences impart on pressure difference, and using velocity change ΔV during Δt and valve thickness L, B may be written as ΔV×L/Δt.

ΔP=A×V²+2×A×B (2)

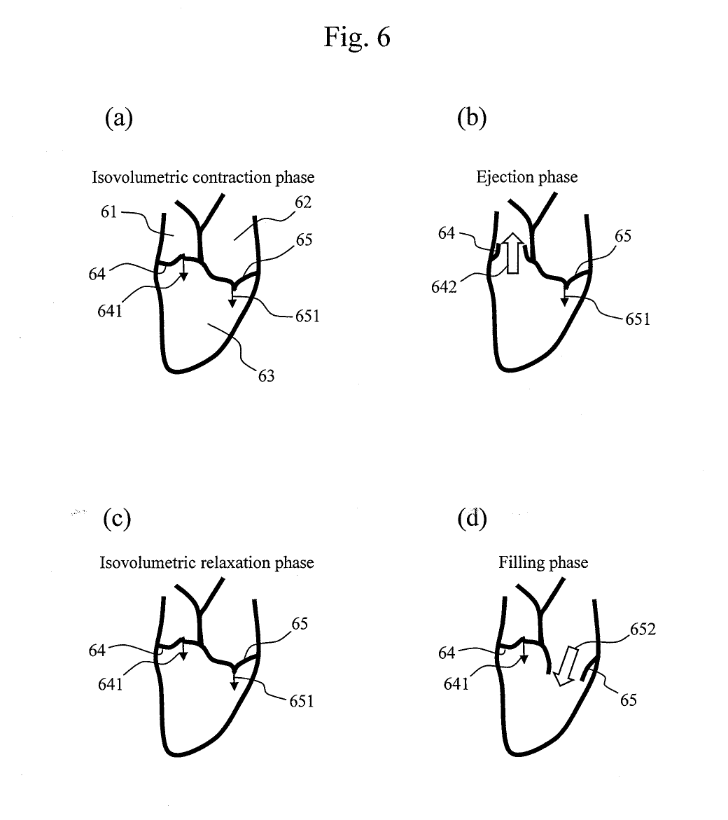

Next, the calculation method for when the valve is open will be discussed. When the valve is open, the simplified Bernoulli principle, where the valve forward flow velocity is substituted into Equation (1), does not hold. The reason for this will be using FIG. 7. When Bernoulli's principle is applied to a valve regurgitant flow, this may be represented with a simplified model such as that in FIG. 7(a). Here, 81a denotes an aorta part, 82a an aortic valve regurgitant outflow part, and 83a the left ventricle. Assuming (Pₐ₁, Vₐ₁, Aₐ₁), (Pₐ₂, Vₐ₂, Aₐ₂), and (Pₐ₃, Vₐ₃, Aₐ₃) are sets of pressure P, velocity V and sectional area A of the site at the respective locations, the following equation holds true under Bernoulli's principle, where ρ is a constant representing blood density.

Pₐ₁/ρ+Vₐ₁²=Pₐ₂/ρ+Vₐ₂²=Pₐ₃/ρ+Vₐ₃² (3)

By utilizing the law of conservation of mass, which states that flow rate Qa, which is the product of velocity and sectional area, is constant regardless of location, the following equation holds true.

Qa=Vₐ₁×Aₐ₁=Vₐ₂×Aₐ₂=Vₐ₃×Aₐ₃ (4)

Here, in order to find the pressure difference between the aorta and the left ventricle, i.e., Pₐ₁−Pₐ₃, from a valve regurgitant flow, an assumption that exit area Aₐ₂ of the aortic valve regurgitant outflow part 82a is sufficiently small in comparison to aorta sectional area Aₐ₁ or left ventricle sectional area Aₐ₃ becomes necessary.

By imposing this assumption, the velocities at the aorta part and left ventricle may be disregarded by virtue of the above-mentioned condition of constant flow rate.

Vₐ₁=Vₐ₃=0 (5)

Further, jet flows whose velocity is equal to or less than 30% of the speed of sound are characteristic in that the pressure at the flow path exit is equal to external pressure. Thus, by regarding regurgitant flow 84a in FIG. 7(a) as a jet flow in the direction of the left ventricle, aortic valve regurgitant outflow part Pₐ₂ and Pₐ₃ may be considered equal.

Pₐ₂=Pₐ₃ (6)

Thus, Bernoulli's principle may be written as follows, and this is how pressure difference is calculated from a regurgitant flow using Bernoulli's principle.

Pₐ₁=Pₐ₃=ρ×(Vₐ₂²)/2 (7)

It is noted that Equation (7) is an equation that assumes a steady state. If unsteady influences are to be taken into account, pressure difference may be calculated as in the following equation by using the discrete unsteady Bernoulli equation.

$${P}_{a 1} - {P}_{a 3} = ρ {\int }_{a 2}^{a 1} \frac{\partial V}{\partial t} d x + ρ \frac{{V}_{a 2}^{2}}{2} \qquad ( 8 )$$

However, when the valve is open, the above-mentioned assumption that exit area Aₐ₂ of the aortic valve regurgitant outflow part 82a is sufficiently small in comparison to aorta sectional area Aₐ₁ or left ventricle sectional area Aₐ₃ does not apply, and a model such as that in FIG. 7(b) is anticipated. Here, 81b denotes an aorta part, 82b an aortic valve regurgitant outflow part, and 83b the left ventricle. Assuming (P_{b1}, V_{b1}, A_{b1}), (P_{b2}, V_{b2}, A_{b2}), and (P_{b3}, V_{b3}, A_{b3}) are sets of pressure P, velocity V and sectional area A of the site at the respective locations, then Bernoulli's principle and the law of conservation of flow rate Qb may be written as follows.

P_{b1}/π+V_{b1}²=P_{b2}/ρ+V_{b2}²=P_{b3}/ρ+V_{b3}² (9)

Qb=Vb₁×A_{b1}=V_{b2}×A_{b2}=V_{b3}×A_{b3} (10)

In particular, since pressure P_{b2} at the valve is unknown, pressure difference P_{b1}−P_{b3} cannot be calculated using valve forward flow velocity V_{b2} based on the law of conservation presented above.

As such, by using fluid motion equations that hold true even when the valve is open, the pressure difference while the valve is open may be calculated. For the motion equation, the Navier-Stokes equation

∇P=−ρ×(∂Vᵢ/∂t+Vⱼ×∂Vᵢ/∂xᵢ)+μ×∂²Vᵢ/∂xᵢ∂xⱼ (11),

which represents the law of conservation of momentum in a fluid, may be used, where Vᵢ is the i-direction component of blood velocity vector V at arbitrary location X within a cardiac chamber, ∇P the pressure gradient at location X mentioned above, ρ a constant representing blood density and that is equal to or greater than 1000 kg/m³ but equal to or less than 1100 kg/m³, and μ a constant representing blood viscosity and that is equal to or greater than 3500 Kg/m/s but equal to or less than 5500 Kg/m/s.

Alternatively, the following Euler equation, which is a simplified version of the Navier-Stokes equation, may be used.

∇P=−ρ×(∂Vᵢ/∂t+Vⱼ×∂Vᵢ/∂xᵢ) (12)

In order to calculate pressure gradient ∇P through the equations discussed above, a velocity space distribution of the fluid is required. For the method of obtaining spatial velocity, a method that obtains a three-dimensional velocity distribution is preferable. This may be attained by using a probe that is capable of three-dimensional imaging. A flow field may be obtained three-dimensionally by three-dimensionally obtaining an image of blood cells or of a pre-administered tracer, e.g., a contrast agent, etc., in blood, and tracking this over time. Three-dimensionality in the context of this method refers to the derivation of velocity information for two or more points in each of three independent directions with respect to a point along a straight line or curve between two points for which pressure difference is to be calculated. In other words, if reference point X₀ and location X₁ are set in a given plane, it may be an imaging region on a slice obtained by giving this plane some thickness. When a contrast agent is administered to a living organism, the invasiveness with respect to the living organism is no longer non-invasive, and becomes minimally invasive.

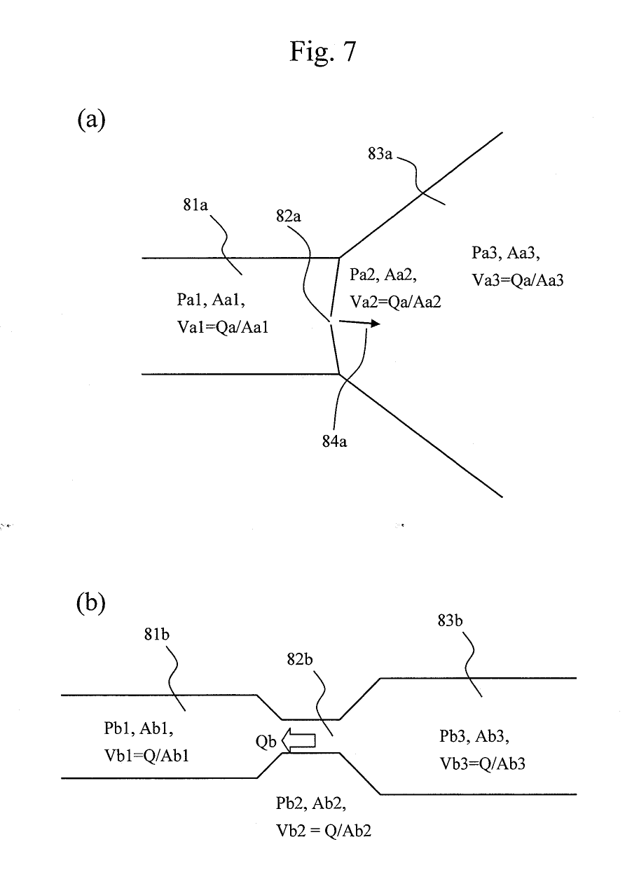

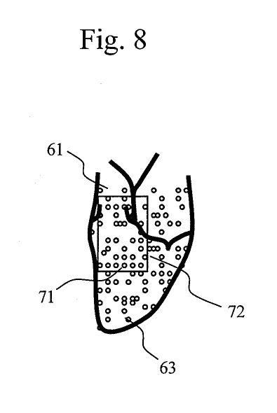

In addition, with respect to details of a velocity obtaining method that uses a tracer, simplified two-dimensional illustrative diagrams are shown in FIG. 8 and FIG. 9. FIG. 8 shows a tracer 71 being imaged within the heart including the left atrium 63. As enlarged views of an imaging region (region of interest: ROI) 72 for which velocity is to be calculated, a view imaged at given time t is shown in FIG. 9(a), and a view imaged at time t+∇t that follows by short period ∇t in FIG. 9(b). It is also possible to track the behavior of individual tracers in order to obtain spatial velocity information. However, for the present case, a method in which velocity is calculated by separating the ROI of an imaging region at a given time in a grid-like fashion and tracking the tracer image pattern within each grid will be described with respect to grid 721. By searching the image in FIG. 9(b) for the image pattern of grid 721 in FIG. 9(a) and finding corresponding grid 722, it is possible to calculate the movement amount for grid 721. Assuming this movement amount is R, the velocity of grid 721 may be calculated as R/∇t. By similarly calculating velocity for all grids, spatial velocity vectors such as those in FIG. 9(c) are calculated. Further, besides the above-discussed pattern matching of gridded particle images, spatial velocity vectors may also be calculated by performing pattern matching for individual particles.

In addition, as another method for finding a velocity space distribution, there is a method that utilizes the Doppler effect. Further, it may also be a method that utilizes the Doppler effect and calculates, using the stream function, a velocity vector based on a velocity field. The only velocity information that is calculable through the Doppler effect is the projected component of the ultrasonic projection direction of a velocity vector indicated with a vector. Thus, when the Doppler effect is utilized, angular correction is necessary, and the ultrasonic projection direction component of the velocity vector becomes a source of error. In addition, since the stream function introduces an assumption of a two-dimensional flow field, its use is restricted. For this reason, it may be said that a method that tracks a tracer and calculates a flow field three-dimensionally is optimal.

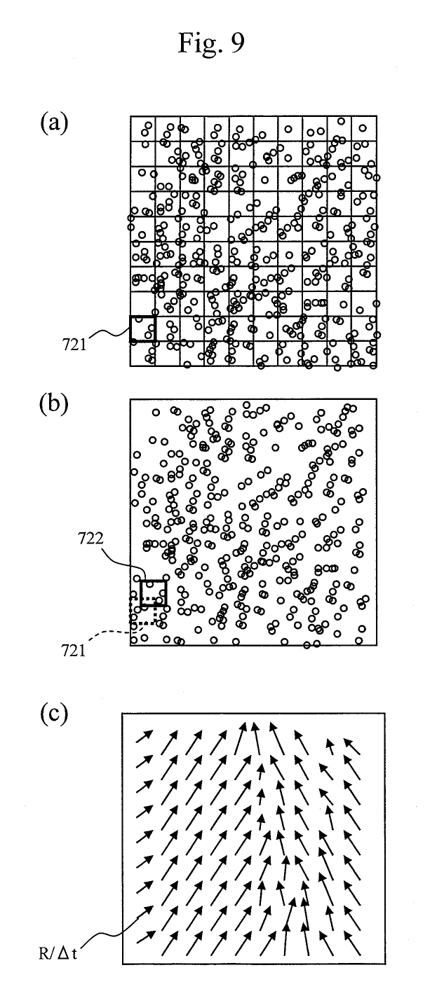

Thus, pressure difference may be calculated not only when the valve is closed but also when the valve is open, and pressure difference may be calculated between a plurality of points at any given heartbeat time phase. A pressure difference contour diagram is shown in FIG. 10. FIG. 10 shows a spatial distribution of pressure calculated from spatial velocity vectors such as those in FIG. 9(c).

Next, the process at the blood pressure difference computation part 1523 will be discussed. If the pressure gradient at location X inside a cardiac chamber is to be calculated, the blood pressure difference computation part specifies arbitrary path L that links reference point X₀ and location X₁, and calculates pressure gradients with respect to discrete path locations L₁, L₂, L₃ . . . , L_{N} along path L, where N is an arbitrary integer. If there is no valve along path L, or if the valve is open, the sum of the products of the pressure gradients at locations L₁, L₂, L₃, . . . , L_{N} for which pressure gradients have been calculated and the distances among the discrete path locations is taken to be the pressure difference between reference point X₀ and location X₁. In addition, if a valve exists at L_{M} along path L and is closed, pressure difference is calculated based on Bernoulli's principle, and the sum of the products of the calculated pressure gradients at locations L₁, L₂, L₃, . . . , L_{N} and the distances among the discrete path locations is taken to be the pressure difference between reference point X₀ and location X₁. Here, spatial pressure difference may also be calculated by substituting 0, or a constant equal to or greater than −1 mmHg/cm but equal to or less than 1 mmHg/cm, for the pressure gradient of a region with a small flow rate. In addition, when the valve is open, given the advantages of reduced complexity, pressure difference may also be calculated utilizing Bernoulli's principle. By means of the blood pressure difference computation part above, the pressure difference at an arbitrary location between cardiac chambers or between blood vessels may be calculated.

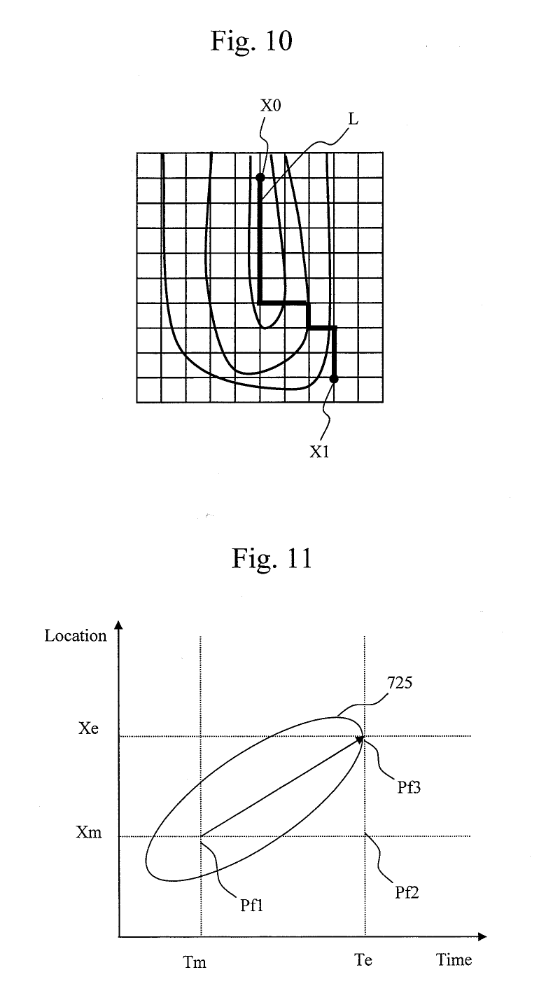

Further, pressure difference may be calculated based on in-flow blood velocity propagation velocity. In-flow blood velocity propagation velocity W may be calculated through Doppler M-mode which represents the change in blood velocity over time. As shown in FIG. 11, blood flowing into the aorta from the left ventricle was measured in Doppler M-mode. Tₘ denotes the time at which the highest flow velocity was observed, Xₘ the location coordinate, and P_{f1} this point. The inner side of a contour line 725 indicating a region that is K % of the highest flow velocity will be referred to as a high-speed region. In the present example, K was defined as 70. However, K may be any value between 40 and 95. Tₑ denotes the time at the other end of the contour line 725 and Xₑ this location. P_{f3} denotes this point. The gradient of the vector between P_{f1} and P_{f3} is in-flow blood velocity propagation velocity W. Assuming that V_{f1}, V_{f2} and V_{f3} denote the respective flow velocities at locations P_{f1}, P_{f2} and P_{f3} respectively indicated by coordinate locations (Tₘ, Xₘ), (Tₑ, Xₘ) and (Tₑ, Xₑ), pressure ΔP between the left ventricle and the aorta may be calculated as follows.

ΔP=−ρ×(W×(V_{f2}−V_{f1})+V_{f2}×(V_{f3}−V_{f2})) (13)

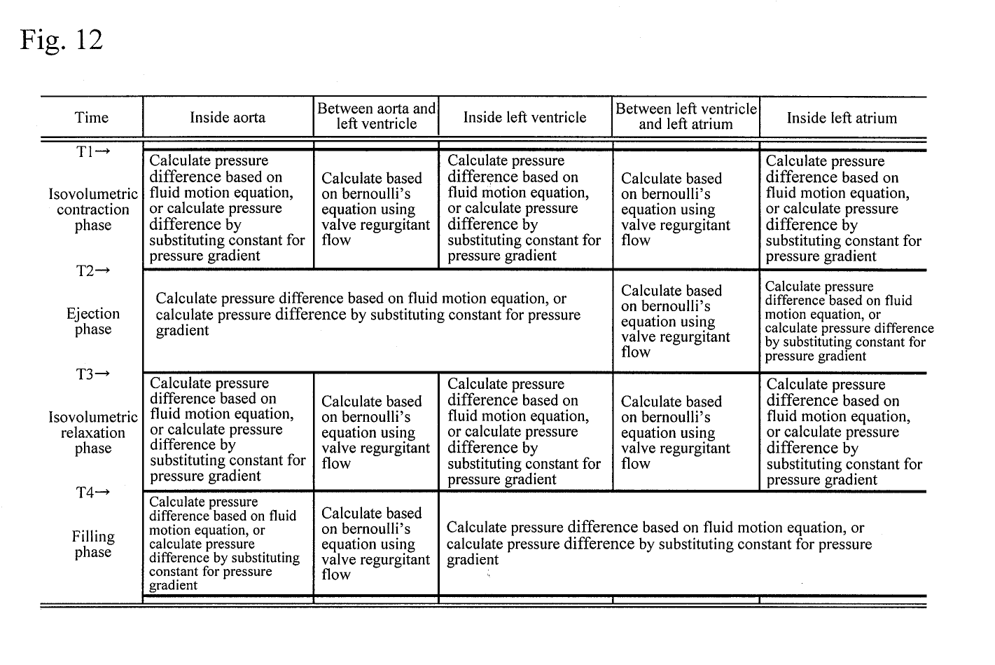

FIG. 12 is a figure in which method selection is sorted by time and location while incorporating the switching timing.

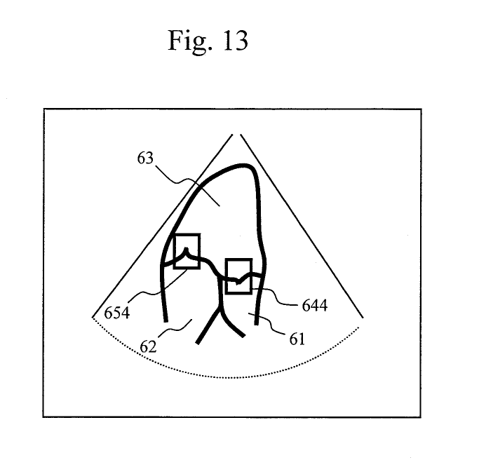

In addition, the regurgitant flow detection in step 134 may be performed by monitoring blood flow near the valve. By setting one of a mitral valve ROI 654 and an aortic valve ROI 644 near the valves as shown in FIG. 13, a valve regurgitant flow may be detected through a detection method that utilizes the Doppler effect, or through a method that tracks blood cells, or a pre-administered tracer, e.g., a contrast agent, etc., in the regurgitant blood flow by image recognition.

Next, details of step 14 in FIG. 2 will be discussed. By subtracting, from the aortic pressure time phase (which will be referred to as pressure waveform) calculated in step 12, a pressure difference waveform that is the fluctuation over time in the pressure difference obtained in step 13, the pressure waveform with respect to location X₁ is derived (S14). The pressure difference waveform between the aorta and the left ventricle may be represented as in curve 532 in FIG. 5(b), and the pressure difference waveform between the left ventricle and the left atrium as in curve 531. In addition, the pressure difference waveform between the aorta and the left atrium may also be calculated by adding up the pressure difference between the aorta and the left ventricle and the pressure difference between the left ventricle and the left atrium. The pressure waveform of the radial artery as converted into the aorta pressure waveform 511 with a transfer function is exchanged. Since phase information is also included in the transfer function, if, at the time of computation, there is any misalignment between the time phase of the calculated aortic pressure and the time phase of the pressure difference, there is a possibility that the time phase may become misaligned. By correcting this, absolute pressure calculation with good precision becomes possible. Time phase correction may be carried out by performing waveform pattern matching. By way of example, it is possible to compute the cross-correlation between the aortic pressure waveform 511 and the pressure difference waveform between the aorta and the left atrium, and detect the misalignment between time phases indicating the greatest value. By correcting time phase misalignment, it becomes possible to compute the absolute pressure at location X with good precision.

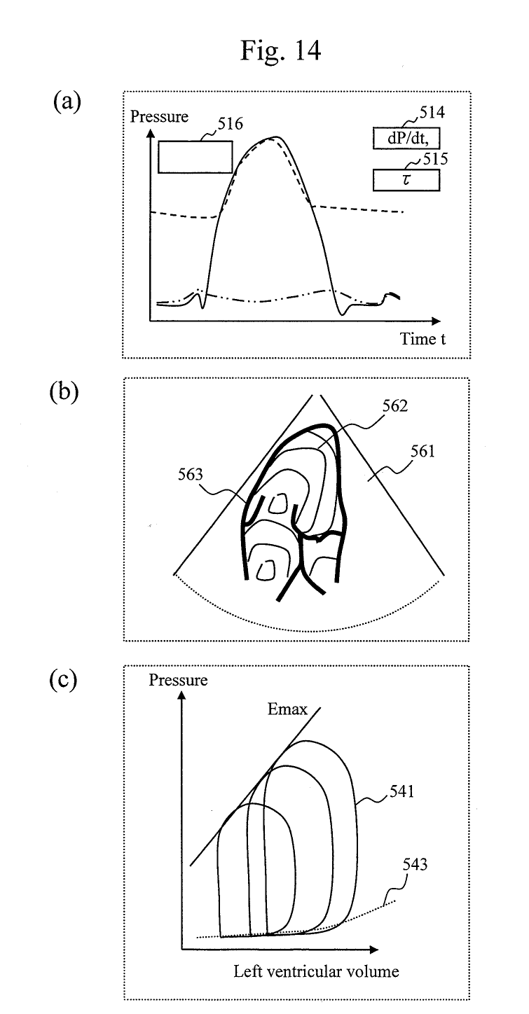

Details of the display part 17 are discussed below. The display part 17 displays the absolute pressure calculated by the absolute pressure computation part 154 with respect to one or more spatial locations, or at a given time, or at one or more of some consecutive times. The above-mentioned absolute pressure may also represent, of an absolute pressure spatial distribution calculated at the absolute pressure computation part 154, the average value, the greatest value, or the smallest value with respect to a plurality of spatial locations desired by the examiner. Display examples are shown in FIG. 14. FIG. 14(a) shows fluctuations in absolute pressure over time. FIG. 14(b) shows a spatial distribution of pressure in a given time phase. Time phase changes of FIG. 14(b) may be displayed as a moving image as well. In addition, based on the image formed at the shape image formation part 151, it may be superimposed on an organ image.

In addition, the absolute pressure computation part 154 of the present invention further comprises an index analysis part. Based on the absolute pressure calculated by the absolute pressure computation part, the index analysis part may calculate dP/dt, which is a physical quantity representing a temporal differential value, and/or time constant τ from when a relaxed state of the left ventricle is approximated with an exponential function, and display one or both of dP/dt and τ with respect to an entire heartbeat or a portion of its duration at display parts 514, 515 as shown in FIG. 14(a). In addition, the progress status of such processes as the various steps shown in FIG. 2 may also be displayed in box 516 in FIG. 14(a).

Further, based on the shape image formed by the shape image formation part 151, the index analysis part may detect the volume of the left ventricle at a plurality of times, and display, on the display part 17, a pressure-volume relationship diagram, which is a diagram that plots, with respect to a space with two or more dimensions and that has an axis representing heart volume and an axis representing absolute pressure, left ventricular volumes at a plurality of times and absolute pressures at a plurality of times calculated by the absolute pressure computation part 154. As shown in FIG. 14(c), in addition to the pressure-volume relationship curve 541, the pressure-volume relationship diagram may also display Eₘₐₓ, which is the gradient of the end-systolic pressure-volume relationship, and an end-diastolic pressure-volume relationship curve 543, which represents the relationship between end-diastolic pressure and volume.

The left ventricular volume may be calculated by the Pombo method or the Teichholz method, which assume the left ventricle to be a spheroid and perform calculations based on the inner diameter of the left ventricle as obtained from a two dimensional image. Alternatively, it may be measured directly by three-dimensionally imaging the shape of the heart.

End-diastolic pressure P_{LV}^{ED} may be calculated as follows.

P_{LV}^{ED}=P_{Ao}−ΔP^{Op} (14)

Here, P_{Ao} is the aortic pressure from end-diastole up to when the aortic valve opens. Since aortic pressure varies little from end-diastole up to when the aortic valve opens, P_{Ao} may assume any given value, or an average value, of aortic pressure from end-diastole up to when the aortic valve opens. In addition, ΔP^{Op} is the pressure difference between the left ventricle and the left atrium while the aortic valve is open, and may be calculated from the mitral valve regurgitant flow while the aortic valve is open by using the law of conservation of momentum and Bernoulli's principle as expressed by, for example, Equations (1), (2), (8), etc.

### LIST OF REFERENCE NUMERALS

1 device main body
2 ultrasonic probe
3 pressure sensor

1. An ultrasonic imaging device comprising:
an ultrasonic probe that transmits/receives an ultrasonic wave to/from an examinee;
a signal processing part that processes a reflected echo signal received by the ultrasonic probe and a blood pressure signal measured from the examinee;
a display part that displays a result of the signal processing as an image; and
an input part that sets a predetermined point with respect to the image displayed on the display part, wherein
the signal processing part comprises:
a reference pressure computation part that computes, from the blood pressure signal, an absolute reference pressure at a reference point near a predetermined point in a blood flow in a body;
a spatial pressure difference calculation part that calculates a spatial pressure difference between the reference point and an absolute reference pressure calculation location computed by the reference pressure computation part; and
an absolute pressure computation part that calculates an absolute pressure of the pressure calculation location based on the absolute reference pressure and the spatial pressure difference.

2. The ultrasonic imaging device according to claim 1, wherein the spatial pressure difference calculation part comprises:
a blood velocity computation part that detects a blood velocity between the reference point and a specified pressure calculation location based on the ultrasonic signal; and
a blood pressure difference computation part that calculates a spatial pressure difference between the reference point and the pressure calculation location based on the blood velocity.

3. The ultrasonic imaging device according to claim 1, wherein
the spatial pressure difference calculation part comprises a heartbeat time phase detection part that detects a heartbeat time phase, and
the spatial pressure difference is calculated through varying calculation methods in accordance with the time phase detected by the heartbeat time phase detection part.

4. An ultrasonic imaging device comprising:
an ultrasonic probe that transmits/receives an ultrasonic wave;
a pressure sensor that non-invasively detects an artery pressure;
a signal processing part that processes an ultrasonic signal received by the ultrasonic probe and a pressure signal obtained by the pressure sensor; and
a display part that displays a result of the signal processing, wherein
the signal processing part comprises:
a shape image formation part that forms an organ shape image from the ultrasonic signal;
a reference pressure computation part that converts the artery pressure into an absolute reference pressure of a given time phase with respect to a reference point inside the heart or near the heart;
a spatial pressure difference calculation part that calculates a spatial pressure difference between the reference point and a pressure calculation location inside the heart; and
an absolute pressure computation part that calculates an intracardiac absolute pressure using the reference pressure and the spatial pressure difference, and
the spatial pressure difference calculation part comprises:
a heartbeat time phase detection part that detects a heartbeat time phase;
a blood velocity computation part that detects a blood velocity based on the ultrasonic signal; and
a blood pressure difference computation part that calculates a pressure difference based on the blood velocity.

5. The ultrasonic imaging device according to claim 4, wherein the blood pressure difference computation part calculates a pressure difference between the aorta and the left ventricle or between the left ventricle and the left atrium using Bernoulli's principle based on a regurgitant flow velocity of the aortic valve or the mitral valve.

6. The ultrasonic imaging device according to claim 4, wherein the blood velocity computation part detects a blood velocity inside a cardiac chamber, and the blood pressure difference computation part calculates a pressure gradient with respect to a location inside a cardiac chamber based on the law of conservation of momentum in a fluid.

7. The ultrasonic imaging device according to claim 4, wherein the blood pressure difference computation part calculates a pressure difference between the aorta and the left ventricle or between the left ventricle and the left atrium while assuming a pressure gradient inside a cardiac chamber to be a constant equal to or greater than −1 mmHg/cm but equal to or less than 1 mmHg/cm.

8. The ultrasonic imaging device according to claim 4, wherein, based on Bernoulli's principle, a pressure difference between the aorta and the left ventricle is calculated from an aortic valve forward velocity, and a pressure difference between the left ventricle and the left atrium is calculated from a mitral valve forward flow velocity.

9. The ultrasonic imaging device according to claim 4, wherein the blood pressure difference computation part changes its processing method between a case where a valve exists and is closed between the reference point and the pressure calculation location, and a case where no valve exists, or a valve exists but is open, between the reference point and the pressure calculation location.

10. The ultrasonic imaging device according to claim 9, wherein the time at which the processing method is changed is one or a plurality of times that serve as boundaries between an isovolumetric contraction phase, an ejection phase, an isovolumetric relaxation phase, and a filling phase.

11. The ultrasonic imaging device according to claim 4, wherein the reference point is within the aorta or the left ventricle, and the pressure calculation location is in the left ventricle or the left atrium.

12. The ultrasonic imaging device according to claim 10, wherein the heartbeat time phase detection part detects a time for changing the process.

13. The ultrasonic imaging device according to claim 1, wherein the display part displays, with respect to the pressure calculation location calculated by the absolute pressure computation part, a pressure at a predetermined time or a change in pressure over time.

14. The ultrasonic imaging device according to claim 1, further comprising an index analysis part, wherein
based on the absolute pressure calculated by the absolute pressure computation part, the index analysis part calculates (dP/dt), which is a physical quantity representing a temporal differential value, and/or time constant τ from when a relaxed state of the left ventricle is approximated with an exponential function, and
the display part displays the physical quantity (dP/dt) and/or time constant τ.

15. The ultrasonic imaging device according to claim 14, wherein
based on the shape image formed by the shape image formation part, the index analysis part detects a left ventricular volume, which is the volume of the left ventricle, at a plurality of times, and
a pressure-volume relationship diagram and/or Eₘₐₓ is/are displayed, the pressure-volume relationship diagram being a diagram that plots, with respect to a two-dimensional space having an axis representing heart volume and an axis representing absolute pressure, the left ventricular volumes at the plurality of times and the absolute pressures calculated by the absolute pressure computation part at the plurality of times, and Eₘₐₓ being the gradient of an end-systolic pressure-volume relationship with respect to the pressure-volume relationship diagram.
